(19) United States
(12) Patent Application Publication
(10) Pub. No.: US 2019/0069899 A1
(43) Pub. Date: Mar. 7, 2019

(54) POSTPARTUM UTERINE EXTERNAL COMPRESSION WRAP

(71) Applicant: Cook Medical Technologies LLC, Bloomington, IN (US)

(72) Inventors: Andrew P. Isch, West Lafayette, IN (US); Victor Havill, Bloomington, IN (US); Angela R. Dowell, Lafayette, IN (US)

(73) Assignee: Cook Medical Technologies LLC, Bloomington, IN (US)

(21) Appl. No.: 16/117,914

(22) Filed: Aug. 30, 2018

Related U.S. Application Data
(60) Provisional application No. 62/553,228, filed on Sep. 1, 2017.

(51) Int. Cl. A61B 17/122; A61B 17/42

(52) U.S. Cl. CPC A61B 17/122; A61B 2017/12004; A61B 17/42

(57) ABSTRACT

An external uterine compression wrap has a sheet like structure with an unfolded configuration and a folded configuration. The sheet is configured to be wrapped around the uterus and secured in the folded configuration to provide external compression to the uterus to treat postpartum hemorrhage. The wrap may include securement members attached to the sheet at one end that extend through corresponding passageways at the opposite end, such that the sheet when folded may be retained in the folded configuration after the securement members have been secured. The wrap may include a securement member in the form of a suture that extends along at least a portion of the perimeter of the sheet through spaced apart holes, and the suture may be pulled to cinch the sheet around the uterus in a folded configuration.

### CROSS-REFERENCE TO RELATED APPLICATIONS

This application claims priority from U.S. Provisional Application No. 62/553,228, filed Sep. 1, 2017, the entirety of which is hereby fully incorporated by reference herein.

### BACKGROUND

### 1. Technical Field

The present disclosure relates to medical devices. More particularly, the disclosure relates to a compression wrap for treating postpartum hemorrhage.

### 2. Background Information

Blood loss during childbirth is a common occurrence in women. Women undergoing c-sections will typically lose more blood than women that deliver vaginally. A normal occurrence during childbirth includes the separation of the placenta from the uterus, which results in open blood vessels in the uterus. In many instances, natural contraction of the uterus after childbirth will cause the blood vessels to close.

In some cases, women may continue to experience bleeding in the uterus that is not resolved by typical uterine contraction. Instances of excessive bleeding can be referred to as a postpartum hemorrhage, or “PPH.” PPH must be treated quickly to limit the amount of blood loss to the woman.

One method of treating PPH involves the physician massaging or manually compressing the uterus with her hands to stop the excessive bleeding. However, this approach is only a short term solution and may not be effective for many instances of PPH.

Another approach for treating PPH includes the use of an inflatable balloon inserted into the uterus and inflated to provide an outward force on the internal wall of the uterus. However, this approach does not provide external compression of the uterus.

To provide external compression of the uterus, one common approach is the B-Lynch suture method. The B-lynch method is a form of compression on the exterior surface of the uterus, and is commonly used in addition to the internal balloon, to result in both internal and external compressive forces to stop the bleeding.

The B-Lynch method includes the use of sutures that are wrapped around the exterior of the uterus. More particularly, the surgeon accesses the uterus through an abdominal incision, which may be the incision from a c-section or a new incision. The suture will puncture the uterine tissue and wrap vertically around the fundus and back down the uterus, and then will turn horizontally and then vertically again to wrap around the opposite lateral side of the uterus. The suture will then be put in tension, thereby providing a vertical compression on each lateral side of the uterus.

However, this approach can cause damage to the uterus due to the tension in the suture and the thin diameter of the suture, where the forces are concentrated on the suture. Further, this approach can result in uneven compressive forces, where the external compression is limited to the location of the sutures, with little compressive force applied to the area between the sutures.

Another risk in this procedure is the formation of uterine synechiae, or adhesions, after placement of the compressive sutures. Further complications can include formation of hematoma, pyometra, localized areas of uterine necrosis/scarring along the suture placement areas, and full thickness defects of the fundus.

Accordingly, improvements can be made in treating PPH.

### SUMMARY

The present disclosure provides an apparatus for providing compression to an internal body organ. The apparatus includes a flexible body in the form of a sheet and defining a longitudinal axis, the sheet defining a first surface and a second surface, the first and second surfaces disposed on opposite sides of the sheet, wherein the body is configured to be wrapped around an internal organ for providing a compressive force to the outer surface of the organ. At least one securement member is attached to the sheet, the at least one securement member extending longitudinally away from a first end of the sheet in a first direction. At least one passageway, or eyelet, extends through the first and second surfaces of the sheet at a second end of the sheet that is opposite the first end, each of the at least one passageway sized and configured to receive a corresponding one of the at least one securement member.

The sheet includes a folding axis oriented transverse to the longitudinal axis of the sheet. The sheet has a longitudinal length that is greater than a width of the sheet measured along the folding axis. The sheet has an unfolded configuration and a folded configuration, wherein the sheet is configured to be folded over itself to the folded configuration such that a portion of the first surface at the second end faces a portion of the first surface at the first end. In the folded configuration, the at least one securement member extends through the at least one passageway in a direction through the first surface and out of the second surface.

In one form, the sheet includes a secured configuration, where the securement members are secured to each other or to the sheet, and the sheet is prevented from being unfolded. In one form, the securement members are secured to each other via tying of the securement members to each other. In another form, the securement members are secured to each other via suturing to each other or suturing to the wrap.

In one form, the securement members include a pointed end capable of piercing muscle or connective tissue within the body. In one form, the pointed end comprises an attached needle. In another form, the pointed end is defined by a molded end of the securement member.

In one form, the sheet comprises multiple layers. In another approach, the sheet is woven or knitted. In one form, the sheet is elastomeric. In one form, the sheet has a contoured shape, and the first surface defines a concave shape.

In one form, the first end of the sheet defines a cutout extending into the first end, and the sheet includes a pair of tapered portions that transition to the securement member, with the tapered portions being located on opposite lateral sides of the cutout and defining the shape of the cutout.

In one form, the first side includes a polymer coating defining a non-slip texture.

In another embodiment, an apparatus for providing compression to an internal body organ includes a flexible body in the form of a foldable sheet and defining a longitudinal axis, the sheet defining an inner surface and an outer surface when folded, the inner and outer surfaces disposed on opposite sides of the sheet, wherein the body is configured to be wrapped around an internal organ for providing a compressive force to the outer surface of the organ. The sheet has an unfolded configuration defining a first end opposite a second end, and a folded configuration where the first end and second end are adjacent each other.

At least one securement member is coupled to the sheet and moveable relative to the sheet to alter the shape of the sheet when the sheet is folded. A plurality of spaced apart holes are disposed through the sheet and along at least a portion of the perimeter of the sheet when unfolded.

The securement member is threaded through the plurality of the holes, wherein the perimeter of the sheet is reduced in response to pulling on the securement member when the sheet is folded.

In one form, in the unfolded configuration, the sheet defines a first end edge at the first end of the sheet, a second end edge at the second end of the sheet, and first and second side edges on opposite lateral sides of the sheet and extending between the first and second end edges, and the plurality of holes extend along at least the side edges and the first end edge.

In one form, the securement member is threaded through the holes that extend along the first end edge and the side edges when unfolded. In one form, in the folded configuration, the securement member extends out of a hole of one of the side edges and back through one of the holes of the first end edge.

In one form, the sheet has a secured configuration with the sheet in the folded configuration, the securement member is threaded through the plurality of holes and cinched, and the securement member is fixedly secured to the sheet such that the securement member is prevented from being uncinched.

In one form, the securement member is tied, clipped, or sutured in place to fix the securement member.

In another form, the sheet is non-degradable and includes a polymer coating on the surface that is configured to contact the organ.

In one form, a method for providing external compression to the uterus is provided. The method includes delivering a flexible sheet having first and second surfaces and a first end and a second end to an exterior surface of the uterus. The sheet includes at least one securement member coupled to the first end of the sheet and extending away from the first end of the sheet, and the sheet further includes at least one passageway extending through the first and second surfaces of the sheet at the second end of the sheet. The method includes positioning the first surface of the sheet against the exterior surface of the uterus and facing the uterus and positioning the second end of the sheet on the anterior side of the uterus. The method further includes wrapping the sheet over the fundus of the uterus and positioning the first end of the sheet on the anterior side of the uterus. The method also includes routing the securement members that are coupled to the first end of the sheet around the base of the uterus and toward the passageways at the second end of the sheet, and extending the securement members through the passageways in a first direction and, in response thereto, drawing the first end of the sheet toward the second end of the sheet. The method includes, in response to drawing the first and second ends of the sheets together, providing external compression from the sheet to the exterior surface of the uterus, and securing the tethers against retraction in a direction that is opposite the first direction to maintain the external compression.

The above described method may be used with the above described apparatuses for providing external compression to a uterus.

### BRIEF DESCRIPTION OF THE DRAWINGS

FIG. 1 illustrates an external compression wrap in the form of a sheet having a pair of tethers extending from a first end, with the sheet in an unfolded configuration, and passageways through the sheet at a second end;

FIG. 2 illustrates the sheet in a folded configuration, with the tethers extending through the passageways;

FIG. 3 illustrates the sheet wrapped around a uterus, with the tethers extending through the passageways and crossing each other and secured to the sheet to secure the sheet around the uterus;

FIG. 4 is a schematic view of the tethers extending through the passageways and tied to each other to secure the sheet;

FIG. 5 illustrates the tethers extending through the passageways and secured to the sheet, with the tips of the tethers being trimmed;

FIG. 6 illustrates the sheet having a concave surface for contacting the exterior surface of the uterus;

FIG. 7 illustrates the sheet having a cutout at the first end and defining tapered transition portions leading to the tethers;

FIG. 8 illustrates the sheet having wing portions extending from the side edges of the sheet;

FIG. 9 illustrates an alternative embodiment of a sheet in an unfolded configuration, the sheet including a plurality of holes extending around the edge and a suture woven through the holes along the edges of the sheet and having free ends extending out of holes at one end of the sheet;

FIG. 10 illustrates the sheet of FIG. 9 in a folded configuration, with the free ends of the suture passing through the holes of the sheet at the opposite end, and the free ends being drawn to produce a drawstring effect on the sheet around the uterus;

FIG. 11 illustrates the sheet in the folded configuration around the uterus, with the free ends of the suture being secured to the sheet; and

FIG. 12 illustrates the free ends of the sutures passing through holes at the opposite end and being tied together.

### DETAILED DESCRIPTION

The present invention provides a postpartum uterine external compression wrap that can be used as an alternative to the B-Lynch procedure. The disclosure provides also for methods of treatment. The materials, methods, and examples disclosed herein are illustrative only and not intended to be limiting. The disclosed figures are not necessarily to scale.

All publications, patent applications, patents, and other references mentioned herein are incorporated by reference in their entirety. Unless otherwise defined, all technical and scientific terms used herein have the same meaning as commonly understood by one of ordinary skill in the art to which this disclosure pertains. In the case of conflict, the present document and definitions will control.

The terms “proximal” and “distal” and derivatives thereof will be understood in the frame of reference of a medical physician using the device. Thus, proximal refers to locations closer to the physician and distal refers to locations farther away from the physician (e.g., deeper in the patient's vasculature).

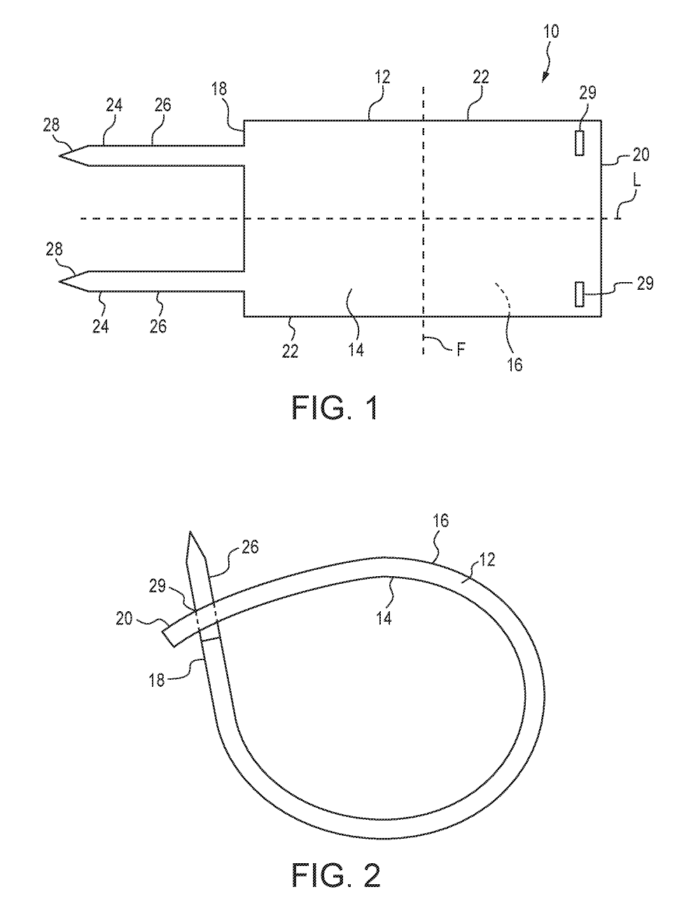

FIG. 1 illustrates a first embodiment of a compression wrap 10. In this embodiment, the wrap is in the form of a sheet 12. As shown in FIG. 1, the sheet 12 is in an unfolded configuration. The sheet 12 is configured to be folded over when placed around the external surface of the uterus, as shown in FIG. 2.

The sheet 12 defines a longitudinal axis L and a folding axis F that is disposed transverse to the longitudinal axis. It will be appreciated that the folding axis F does not necessarily conform to the exact location of where a fold may occur, or that the fold will occur along an axis that is exactly perpendicular to the longitudinal axis.

The sheet 12 includes a first surface 14 and a second surface 16 on opposite sides of the sheet 12. In one form, the first surface 14 may be the surface that is intended to contact to the external surface of the uterus when the sheet 12 is folded over the uterus. However, in another form, either the first or second surface can be the surface that contacts the uterus.

As illustrated, the sheet 12 has a generally rectangular shape, and defines a first end 18 and a second end 20 that are at opposite longitudinal ends of the sheet. The sheet 12 also defines lateral sides 22 that extend longitudinally between the first end and the second end. However, it will be appreciated that other shapes of the sheet could also be used that are not rectangular, such as an oval shape, a polygonal shape, or another shape that may be wrapped around a uterus.

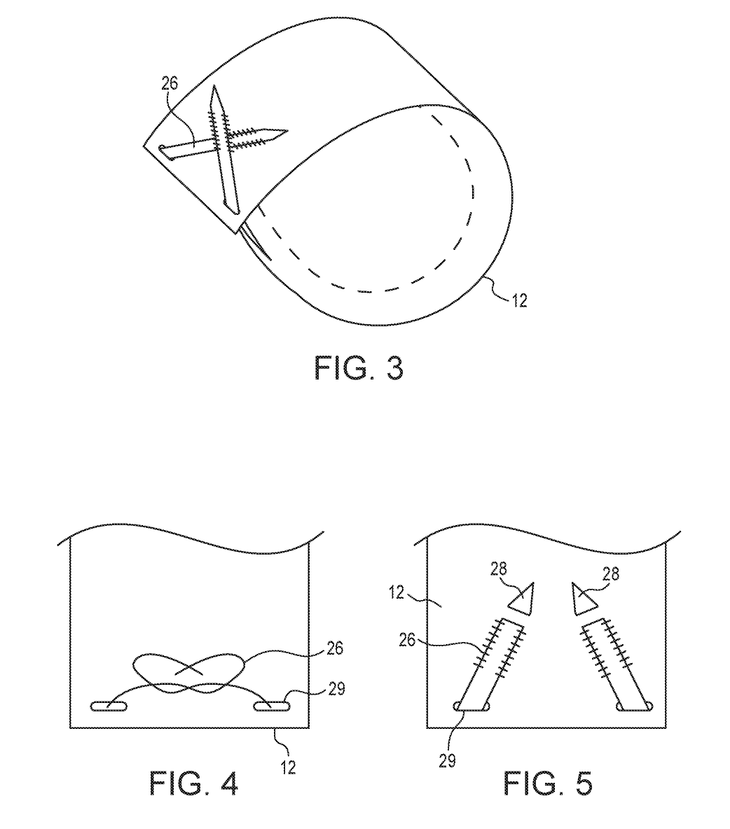

When wrapped around the uterus, as illustrated in FIGS. 2 and 3, the first end 18 and the second end 20 are brought together such that they are adjacent each other, and the sheet 12 is secured in its folded configuration to provide a compressive force around the external surface of the uterus.

The wrap 10 further includes at least one securement member 24 that is attached to the sheet 12. As shown in FIG. 1, the securement member 24 may be in the form of an elongate tether 26. The tether 26 has a strip-like shape, and extends longitudinally away from the first end 18 of the sheet 12. In a preferred form, the wrap 10 includes two tethers 26.

In one form, the tether 26 includes a pointed tip 28. The pointed tip 28 is preferably sharp enough to pierce through the uterus or the connective tissue of the uterus. The pointed tips 28 allow the tethers to act as anchoring structure to anchor the wrap 10 to or around the uterus.

The tethers 26 may be part of a unitary structure with the sheet 12, such that the tethers 26 are extensions of the material used to define the sheet 12. In another form, the tethers 26 may be separate pieces that are attached to the sheet 12 via sutures, bonding, or other known attachment methods.

As shown in FIG. 1, the sheet 12 may further define at least one passageway 29 that extends completely through the material of the sheet 12, such that the passageways 29 extend from the first surface 14 to the second surface 16 of the sheet 12. The passageways 29 are configured to allow the tethers 26 to pass through the sheet 12, as shown in FIGS. 2 and 3. Accordingly, the passageways 29 are preferably at least as wide as the width of the tethers 26, thereby allowing the tethers 26 to pass through the sheet 12. The passageways 29 may also be referred to as eyelets.

The passageways 29 are located at the opposite end of the sheet from the tethers 26. Thus, when the sheet 12 is folded, and the first end 18 and second end 20 of the sheet 12 are arranged in the vicinity of each other, as shown in FIG. 2, the tethers 26 can extend through the passageways 29 to hold the sheet 12 in the folded configuration.

The passageways 29 may be in the form of slots or slits that correspond to the strip-like shape of the tethers 26. Alternatively, the passageways 29 could have other shapes that permit the tethers 26 to pass through. In the case where the passageways 29 correspond to the shape of the tethers 26, the orientation of the passageways can control the orientation of the tether 26.

In one form, the passageways 29 are oriented generally parallel to the edge of the sheet 12 and perpendicular to the longitudinal axis. In another form, the passageways 29 are oriented at an angle relative to the longitudinal axis and at an angle relative to an axis that is perpendicular to the longitudinal axis, or at an angle relative to the edges of the sheet 12.

In the folded configuration, the tethers 26 can extend through the passageways 29 to retain the first end of the sheet 12 to the second end of the sheet 12, thereby securing the sheet 12 in the folded configuration. To maintain the sheet 12 in the folded configuration, the tethers 26 may be secured either to each other or to the sheet 12, as shown in FIGS. 3-5.

For example, the tethers 26 may be tied to each other to secure the wrap 10 in the folded configuration, as shown in FIG. 4. In another form, the tethers 26 may be sutured together, as shown in FIG. 3. In another form, the tethers 26 may be sutured to sheet 12, as shown in FIGS. 3 and 5. In one form, the tether 26 will cross over each other in the secured state, as shown in FIGS. 3 and 4. In another form, the tethers 26 will not cross each other, as shown in FIG. 5. However, in the case where the tethers 26 are tied to each other, the tethers 26 would inherently cross each other such that they can be tied together, as shown in FIG. 4. The tethers 26 may also be secured to each other mechanically, such as via clipping, crimping, clamping, or the like.

After securing the tethers 26, the excess length of the tethers 26 may be cut or trimmed, if desired. Cutting or trimming the tethers 26 can preferably remove the pointed tips to reduce undesired puncturing of adjacent tissue. FIG. 5 illustrates the tips 28 after being cut.

The sheet 12, as described above, can have a generally rectangular shape or other shape. In one form, the sheet 12 is generally flat, such that the first and second surfaces 14 and 16 of the sheet 12 are generally planar.

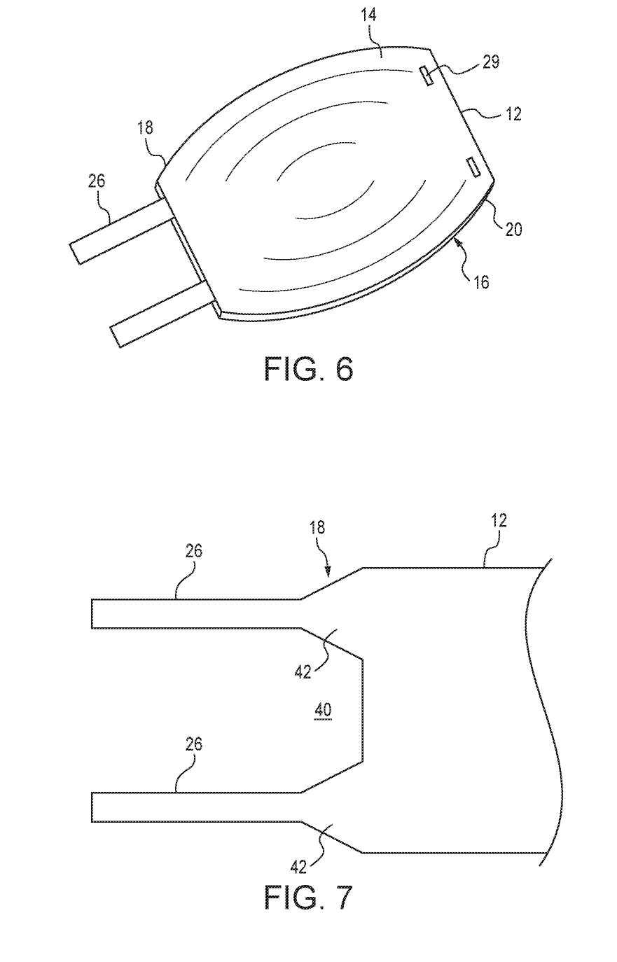

In another form, as shown in FIG. 6, the sheet 12 can have a curved shape, such that one of the surfaces, for example the first surface 14, has a concave shape. Accordingly, the second surface 16 in this example would have a convex shape. The curved shape of the sheet can provide a more tailored and contoured fit around the uterus when the sheet 12 is in the folded condition. The curved embodiment of the sheet 12 can result in a more evenly distributed compressive force when the sheet 12 is folded over the uterus and providing compression. However, it will be appreciated that the flat embodiment of the sheet 12 can still provide an improved compressive force relative to prior methods.

In one form, the sheet 12 can be made from a stretchable or elastomeric material, such that when the sheet 12 is wrapped around the uterus, the sheet 12 may stretch in some areas, thereby becoming curved or concave around the uterus, similar to the shape shown in FIG. 6. In this form, the sheet 12 can maintain a compressive force after the uterus has shrunken relative to its state when the sheet 12 was initially wrapped around the uterus. In this form, a bias against the elasticity of the sheet 12 will return the sheet toward its nominal shape as the uterus shrinks.

The sheet 12 can be in the form of a single layer or multiple layers or plies that are attached together. The sheet 12 can have a graft-like structure, and can be biodegradable, bioresorabable, or non-degradable, depending on the needs of the patient. The sheet 12 can be a woven or knitted material, or a non-woven structure that is blown or extruded, or a non-woven structure that is felted or deposited by vapordeposit, melt spin, or electrospinning. The sheet may include stitching around its edges to reinforce its perimeter. Stitching may also be included around the passageways 29 to reinforce the passageways and prevent tearing when the tethers 26 are pulled tight to secure the sheet 12 in the folded configuration and apply the compressive force.

In one form, the sheet 12 has a graft like structure and made from a material such as PGA, PLA, PLGA, PCL, PVA, PVP, cellulose, Small Intestine Submucosa, or other collagen based material, PGS, or other bioresorbable material. In one form, an optimal disintegration or dissolution, or resorption, time can be between 1 and 60 days, depending on the needs of the patient.

With reference to FIG. 7, the sheet 12 can include cutouts defined by the sheet 12 to allow the sheet to more closely conform to the shape of the uterus. For example, in one form, the sheet 12 can include a cutout 40 at the end of the sheet 12 that includes the tethers 26. In this form, the cutout 40 can have a generally trapezoidal shape extending into the first end 18 of the sheet 12. As a result of the cutout 40, the shape of the sheet 12 as it transitions into the tethers 26 or at the location where the tethers 26 are attached can have a tapered transition portion 42. The transition portions 42 can be used to wrap around the base of the uterus and provide an improved compressive force in this area relative to a sheet 12 with a flat edge and tethers 26 extending from the corners of the sheet 12.

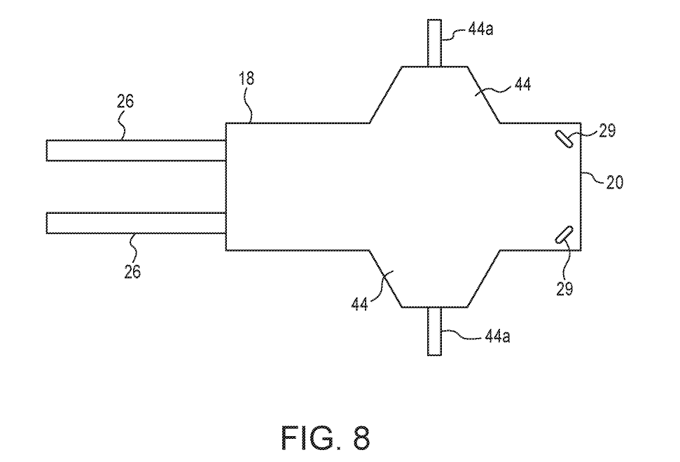

With reference to FIG. 8, the sheet 12 can further include other additional extensions or protrusions, along with additional tethers for these extensions to provide compressive forces in the transverse plane in addition to the compressive forces that are applied in the sagittal plane from the overall sheet 12.

For example, wing portions 44 can extend from the sides 22 of the sheet and laterally outward. Thus, when the sheet 12 is wrapped around the top of the uterus, the wing portions 44 and its tethers 44a can be further wrapped around the sides of the uterus and secured in a manner similar to the tethers 26 described above.

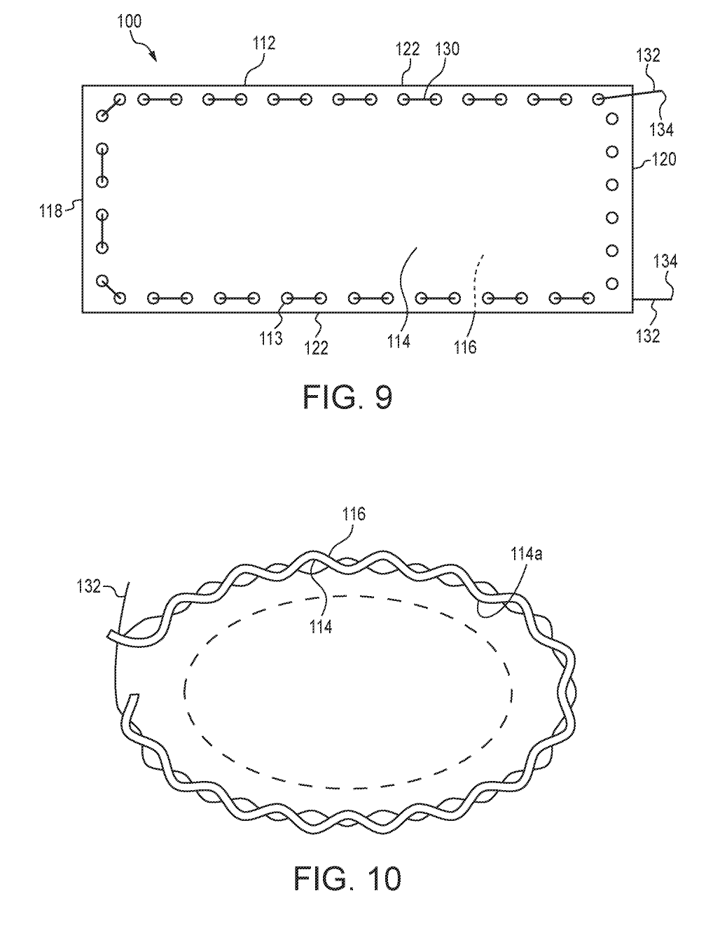

Turning now to FIGS. 9 and 10, in a further embodiment, a wrap 100 may include a sheet 112, similar to the sheet 12, having an unfolded (FIG. 9) and a folded (FIG. 10) configuration, where folding the sheet 112 and wrapping it around the uterus will provide an external compressive force. The sheet 112 may have a rectangular shape when unfolded, similar to the sheet 12. However, the sheet 112 may not include integral or fixedly attached tethers extending from one end. Rather, the sheet 112 may be secured in the folded configuration via a drawstring mechanism, as further described below.

In this form, the sheet 112 includes a plurality of holes 113 extending through the sheet at disposed along the outer perimeter of the sheet 112. As shown, the sheet 112 has a rectangular shape, first and second surfaces 114 and 116, first and second ends 118, and 120, and sides 122. The edges at each end and the sides may include the holes 113 extending along the perimeter. The holes 113 are preferable spaced apart sufficiently to allow for a drawstring effect.

In one form, only one of the first and second ends 118, 120 will have the holes extending fully along the edge or substantially along the edge. In this form, this specific edge may be completely free of holes, or may include holes 113 at the corner, with the middle portion of the edge being free from holes. Put another way, the middle of this edge may have a larger space between the inner most holes.

The sheet 112 may be non-degradable, and in this form would be intended to be removed from the patient. However, the sheet 112 may also be degradable or absorbable. In one form, the material of the sheet 112 can be similar to the LapSac Surgical Tissue Pouch, which comprises a high strength woven fabric that is virtually untearable by hand, allowing a physician a high amount of compressive force without tearing the sheet 112.

The wrap 100 further includes a suture 130 that is routed through the holes 113 in an alternating fashion, as shown in FIG. 9, where portions of the suture 130 lie across the first surface 114 and other portions of the suture 130 lie across the second surface 116. In one form, the suture 130 is preferably routed through the holes 113 that extend along one side 122, through the holes along the first end 118, and through the holes along the other side 122, thereby defining a generally U-shape of the suture 130 routed through the holes 113. The holes 113 along the second end 120 may be initially free from the suture 130, although the suture 130 could subsequently be routed through these holes 113.

The suture 130 preferably includes two free ends 132, where the free ends 132 have a needle-like pointed tip 134, either formed by the suture material, or attached to the suture 130. The pointed tip 134 is arranged such that it can pierce the uterus or the connective tissue adjacent the uterus to aid in securing the wrap 100 around the uterus.

To secure the wrap 100 in the folded configuration shown in FIG. 10, the wrap 100 may be folded into the folded configuration around the uterus, with the free ends 132 at the rear of the uterus. The free ends 132 can be routed forward and through the front side holes 113, where the free ends 132 can be pulled to cause a drawstring effect, thereby cinching the sheet 112 tightly around the uterus to apply a compressive force.

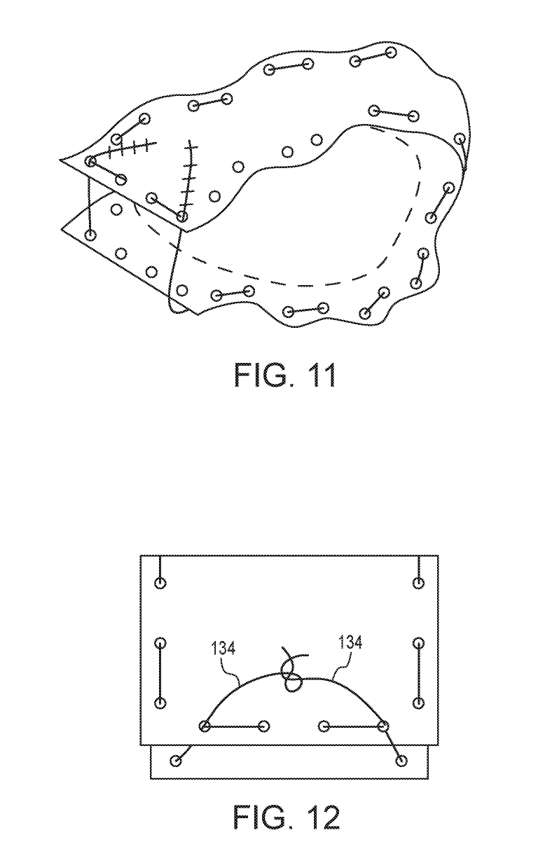

As shown in FIGS. 11 and 12, the free ends 132 of the suture 130, which are longer after cinching than prior to cinching, can then be secured to retain the sheet 112 in the folded configuration. In one form, the free ends 132 may be tied to each other, as shown in FIG. 12. In another form, the free ends can be clipped in place, or sutured to the sheet 112 or the surrounding tissue, as shown in FIG. 11. Once secured, the sheet 112 will apply a compressive force around the uterus.

The sheet 112 can have many of the same features described above with respect to the sheet 12. For example, it can have a curved shape or flat shape. It can have a non-rectangular shape. It can be made from a single layer or multiple layers or plies. It can include reinforcing stitching. The sheet 12 can be a woven or knitted material, or a non-woven structure that is blown or extruded, or a non-woven structure that is felted or deposited by vapordeposit, melt spin, or electrospinning. The sheet 112 can further be elastomeric.

In one form, the sheet 112 may include a polymer coating 114a on the surface 114 that is intended to contact the uterus. The polymer coating may provide a tactile, non-slip texture to the sheet 112 that may be impervious, non-adhesive, and/or impermeable, thereby allowing the sheet 112 to resist slipping across the tissue, thereby increasing the effectiveness of the compression. The polymer coating could also be applied to the sheet 12.

The manner of using the sheet 12 or 112 to compress the uterus has been described above. However, the following description will provide further context.

In the event where external uterine compression is desired, the physician will gain access to the area outside the uterus typically via a previously created cesarean incision. The sheet 12 or 112 will be placed over the uterus, with the surface 14 or 114 placed against the surface of the uterus. The end 20 or 118 will typically be placed at the front or anterior side of the uterus, and the sheet 12 or 112 will be wrapped over the fundus and around the rear or posterior side of the uterus, such that the end 18 or 120 is at the posterior side.

With respect to the sheet 12, the tethers 26 extending from the first end 18 will be routed forward toward the passageways 29 at the second end 20. The tethers 26 may pierce the uterus or the connective tissue around the uterus to anchor the sheet 12 to the uterus, or the tethers 26 may pass around the base of the uterus without piercing the uterus or connective tissue, and the sheet 12 may be anchored by being wrapped around the uterus.

The tethers 26 are passed through the passageways 29 of the sheet 12, thereby bringing the second end 20 and the first end 18 toward each other and wrapping the sheet 12 around the exterior of the uterus to provide external compression. With the tethers 26 extending through the passageways 29, the tethers 26 are then secured to each other via suturing or knotting, or the tethers 26 may be secured to the sheet 12 via suturing. After being secured, any excess portion of the tethers 26 may be trimmed. Other forms of mechanical securement may also be used to secure the tethers 26 to each other, such as a clip, clamp, a crimping mechanism, or the like. The form of securement may be fixed, such that the tethers 26 may be released by later cutting or severing the tether material, or the form of securement may be adjustable, such that the tethers may be released by adjusting or releasing the adjustable form of securement.

In the case of a sheet 12 made of bioresorbable material, the sheet 12 may be left in place after the compression is complete. Alternatively, the sheet 12 can be removed by the physician later.

For the sheet 112, rather than pass the tethers through the passageways 29 as in the sheet 12, the free ends 132 of the suture 130 are routed forward from the rear/anterior side of the uterus and toward the holes 113 that are in the end 118 of the sheet 112. The free ends 132 extending from the second end 120 may pass through the uterine tissue or connective tissue at the base of the uterus as they are routed toward the first end 118. The free ends 132 may be pulled tight, causing a drawstring effect and providing an external compressive force around the uterus by the sheet 112 as the second end 120 is pulled toward the first end 118. With the free ends 132 extending through one or more of the holes 113 at the first end 118, the ends 132 can be secured to each other or the sheet 112 as described above.

At the conclusion of the external compression, the sutures 130 can be released from their securement and the sheet 112 can be removed from the patient.

The above described sheets 12 or 112 may be used along with internal uterine devices that provide internal tamponade, such as uterine balloons, adhesion barrier devices, or the like.

In applying the compressive force by the sheet 12 or 112, the force from the suture 130 or the tethers 26 is generally isolated from the uterus, such that the force is distributed to the exterior of the uterus via the sheet 12 or 112, rather than directly by a suture. By distributing the compressive load, trauma to the uterus can be decreased.

While the present invention has been described in terms of certain preferred embodiments, it will be understood that the invention is not limited to these disclosed embodiments, as those having skill in the art may make various modifications without departing from the scope of the following claims.

1. An apparatus for providing compression to an internal body organ, the apparatus comprising:
a flexible body in the form of a sheet and defining a longitudinal axis, the sheet defining a first surface and a second surface, the first and second surfaces disposed on opposite sides of the sheet, wherein the body is configured to be wrapped around an internal organ for providing a compressive force to the outer surface of the organ;
at least one securement member attached to the sheet, the at least one securement member extending longitudinally away from a first end of the sheet in a first direction;
at least one passageway extending through the first and second surfaces of the sheet at a second end of the sheet that is opposite the first end, each of the at least one passageway sized and configured to receive a corresponding one of the at least one securement member;
wherein the sheet includes a folding axis oriented transverse to the longitudinal axis of the sheet;
wherein the sheet has a longitudinal length that is greater than a width of the sheet measured along the folding axis;
wherein the sheet has an unfolded configuration and a folded configuration, wherein the sheet is configured to be folded over itself to the folded configuration such that a portion of the first surface at the second end faces a portion of the first surface at the first end;
wherein, in the folded configuration, the at least one securement member extends through the at least one passageway in a direction through the first surface and out of the second surface.

2. The apparatus of claim 1, wherein the sheet further includes a secured configuration, where the securement members are secured to each other or to the sheet, and the sheet is prevented from being unfolded.

3. The apparatus of claim 2, wherein the securement members are secured to each other via tying of the securement members to each other or mechanically coupling the securement members to each other.

4. The apparatus of claim 2, wherein the securement members are secured to each other via suturing to each other or suturing to the wrap.

5. The apparatus of claim 1, wherein the securement members include a pointed end capable of piercing connective tissue within the body.

6. The apparatus of claim 5, wherein the pointed end comprises an attached needle.

7. The apparatus of claim 5, wherein the pointed end is defined by a molded end of the securement member.

8. The apparatus of claim 1, wherein the sheet comprises multiple layers.

9. The apparatus of claim 1, wherein the sheet is woven or knitted.

10. The apparatus of claim 1, wherein the sheet is elastomeric.

11. The apparatus of claim 1, wherein the sheet has a contoured shape, and the first surface defines a concave shape.

12. The apparatus of claim 1, wherein the first end of the sheet defines a cutout extending into the first end, and the sheet includes a pair of tapered portions that transition to the securement member, the tapered portions located on opposite lateral sides of the cutout and defining the shape of the cutout.

13. The apparatus of claim 1, wherein the first side includes a polymer coating.

14. An apparatus for providing compression to an internal body organ, the apparatus comprising:
a flexible body in the form of a foldable sheet and defining a longitudinal axis, the sheet defining an inner surface and an outer surface when folded, the inner and outer surfaces disposed on opposite sides of the sheet, wherein the body is configured to be wrapped around an internal organ for providing a compressive force to the outer surface of the organ;
wherein the sheet has an unfolded configuration defining a first end opposite a second end, and a folded configuration where the first end and second end are adjacent each other;
at least one securement member coupled to the sheet and moveable relative to the sheet to alter the shape or size of the sheet when the sheet is folded;
a plurality of spaced apart holes disposed through the sheet and along at least a portion of the perimeter of the sheet when unfolded;
wherein the securement member is threaded through the plurality of the holes, wherein the at least a portion of the perimeter of the sheet is reduced in response to pulling on the securement member when the sheet is folded.

15. The apparatus of claim 14, wherein, in the unfolded configuration, the sheet defines a first end edge at the first end of the sheet, a second end edge at the second end of the sheet, and first and second side edges on opposite lateral sides of the sheet and extending between the first and second end edges, and the plurality of holes extend along at least the side edges and the first end edge.

16. The apparatus of claim 15, wherein the securement member is threaded through the holes that extend along the first end edge and the side edges when unfolded.

17. The apparatus of claim 16, wherein, in the folded configuration, the securement member extends out of a hole of one of the side edges and back through one of the holes of the first end edge.

18. The apparatus of claim 14, wherein the sheet has a secured configuration with the sheet in the folded configuration, and the securement member is threaded through the plurality of holes and cinched, and the securement member is fixedly secured to the sheet such that the securement member is prevented from being uncinched.

19. The apparatus of claim 14, wherein the sheet is non-degradable and includes a polymer coating on the inner surface.

20. A method for providing external compression to the uterus, the method comprising:
delivering a flexible sheet having first and second surfaces and a first end and a second end to an exterior surface of the uterus, wherein the sheet includes at least one securement member coupled to the first end of the sheet and extending away from the first end of the sheet, and the sheet further includes at least one passageway extending through the first and second surfaces of the sheet at the second end of the sheet;
positioning the first surface of the sheet against the exterior surface of the uterus and facing the uterus and positioning the second end of the sheet on the anterior side of the uterus;
wrapping the sheet over the fundus of the uterus and positioning the first end of the sheet on the anterior side of the uterus;
routing the securement members that are coupled to the first end of the sheet around the base of the uterus and toward the passageways at the second end of the sheet;
extending the securement members through the passageways in a first direction and, in response thereto, drawing the first end of the sheet toward the second end of the sheet;
in response to drawing the first and second ends of the sheets together, providing external compression from the sheet to the exterior surface of the uterus; and
securing the tethers against retraction in a direction that is opposite the first direction to maintain the external compression.
